(19) United States
(12) Patent Application Publication
(10) Pub. No.: US 2019/0344089 A1
(43) Pub. Date: Nov. 14, 2019

(54) Electrode Lead with Fixing Device

(71) Applicant: BIOTRONIK SE & Co. KG, Berlin (DE)

(72) Inventors: Detmar Jadwizak, Erkner (DE); Dajana Kaiser, Berlin (DE); Carsten Fruendt, Berlin (DE); Gordon Hillebrand, Berlin (DE)

(21) Appl. No.: 16/405,171

(22) Filed: May 7, 2019

(30) Foreign Application Priority Data
May 9, 2018 (DE) 10 2018 111 178.9

(51) Int. Cl. A61N 1/375; A61N 1/05

(52) U.S. Cl. CPC A61N 1/37518; A61N 1/05; A61N 1/37516

(57) ABSTRACT

A fixing device for fixing an implantable electrode lead in a blood vessel, which fixing device has a main body with a substantially circular cross-section, and at least one resilient fixing element arranged on the main body in a contact region, wherein the fixing element extends in a first direction of extent in an axial direction in respect of the main body, extends away from the main body starting from the contact region, and wherein the fixing element encloses an angle of less than 45-degrees with a tangent running through the contact region. Also provided is an implantable electrode lead comprising this fixing device.

### CROSS-REFERENCE TO RELATED APPLICATIONS

This patent application claims the benefit of and priority to co-pending German Patent Application No. DE 10 2018 111 178.9, filed on May 9, 2018 in the German Patent Office, which is hereby incorporated by reference in its entirety.

### TECHNICAL FIELD

The present invention relates to a fixing device for fixing a longitudinally extended element, in particular in the form of an implantable electrode lead, in a blood vessel. The present invention also relates to an electrode lead that comprises a fixing device of this kind.

### BACKGROUND

Typical examples of electrode leads that are anchored in a blood vessel are electrode leads for cardiac resynchronisation therapy (CRT). These electrode leads normally have an elongate lead body, are usually connectable by means of a connection device, such as a plug, to a pulse generator, and in their distal region have electrodes connected to the lead body for contacting bodily tissue.

A particularly simple fixing technique for avoiding dislocations of electrode leads of this kind in the coronary sinus region is constituted by a wedging of the distal end of the lead body in a vessel. Here, the electrode lead is for example advanced into the branches of the coronary sinus until the distal end of the lead body becomes stuck in the tapering vein and assumes a “wedge position”. In this position, the vein is closed by the electrode lead.

In order to fix an electrode lead in a wedge position, numerous solution approaches have already been proposed. For example, to this end the distal end of the electrode lead can be provided with a surface structure that hinders or prevents the withdrawal of the advanced electrode lead. In order to intensify this effect, the distal end of the electrode lead is alternatively or additionally curved or S-shaped, for example. Furthermore, the distal end of the electrode lead can be configured as a screw made of silicone, such that the electrode can be screwed into the narrowing vessel.

The possibility of secure fixing of the electrode lead by means of a wedge position of this kind, however, is heavily dependent on the geometry (for example the course, the length and the diameter, etc.) of the target vein in the coronary sinus. The end position of the stimulation and/or sensing electrode(s) is therefore usually defined by the geometry of the target vein. Multipolar electrode leads, for example, help in these cases to achieve longitudinally the best-possible location for the therapy. In order to achieve an optimal effect of the stimulation pulses, it is additionally necessary however for the electrodes to be brought as close as possible to the tissue to be stimulated. Here, a fixing variant of the electrode lead that makes it possible, in addition to providing a longitudinally optimal position, to also guide the electrodes as closely as possible to the wall of a blood vessel and to hold them in this position is desirable.

The present invention is directed at overcoming one or more of the above-mentioned problems.

### SUMMARY

On this basis, an object is to create a fixing device which is improved in respect of the above-mentioned problems. On this basis, a further object is to create an electrode lead that is provided with a fixing device of this kind.

At least these problems are solved by a fixing device having the features of claim 1 and by an electrode lead having the features of claim 13. Further embodiments are specified in the corresponding dependent claims and will be described hereinafter.

According to claim 1, a fixing device for fixing an implantable electrode lead in a blood vessel is disclosed, which fixing device has a main body with a circular cross-section, with at least one resilient fixing element arranged on the main body in a contact region, wherein the fixing element extends in a first direction of extent in an axial direction with respect to the main body, wherein the fixing element in a second direction of extent extends starting from the contact region, away from the main body, and wherein the fixing element encloses an angle of less than 45-degrees with a tangent on the main body running through the contact region.

When inserting the fixing device into a blood vessel in which the fixing device is to be anchored, the longitudinal axis of the main body of the fixing device is arranged substantially coaxially with the longitudinal axis of the blood vessel. So that an anchoring is possible, both the edge(s) of the fixing element(s) opposite the corresponding contact region and the main body of the fixing device must be in contact with the vessel wall. In cases in which at least three fixing elements are arranged on the main body, spaced apart at substantially equal distances in the circumferential direction, it is sufficient if the edges of the fixing elements opposite the associated contact region have contact with the wall. Thus, the maximum diameter of a vessel in which the fixing device can be successfully anchored is dependent on the diameter of the main body of the fixing device, and the maximum distance between the contact region between the main body and fixing element and the edge of the associated fixing element opposite the contact region. Since the fixing element is inclined relative to the tangent running through the contact region in question or in some cases runs parallel to this tangent, the fixing element, with reducing diameter of the blood vessel, is pressed in the direction of the main body of the fixing device. The fixing device can hereby be adapted to the particular outer diameter of the blood vessel as it is inserted.

The fixing device is rotatable about the longitudinal axis of the fixing device perpendicular to the circular cross-sectional area. In order to define the directions of rotation, a rotation without wall contact is firstly assumed. Here, a rotation in a first direction of rotation D1 is defined such that the fixing element runs behind the associated contact region. By contrast, in the event of a rotation in a second direction of rotation D2, the fixing element runs ahead of the associated contact region.

It is now assumed that the fixing device is inserted into a blood vessel and that the fixing element has contact with the wall. If the fixing device is rotated about its longitudinal axis in the first direction of rotation D1, a first force acts on the fixing element and presses the fixing element in the direction of the main body of the fixing device.

If, by contrast, the fixing device is rotated about its longitudinal axis in the second direction of rotation D2, a second force acts on the fixing element and deploys the fixing element. As a result of the second force, the fixing element sticks by means of its edge opposite the contact region against the vessel wall, and the fixing element is hereby buckled along the second direction of extent and is bent, such that the fixing element has a convex side and a concave side. Here, the convex side points in the second direction of rotation D2, whereas the concave side points in the first direction of rotation D1. A balance is established depending on the diameter of the blood vessel and the bending moment of the fixing element along the second direction of extent. In this state, the fixing device is tensioned, and the fixing element exerts a force onto the wall of the blood vessel, whereby the fixing device remains fixed in the blood vessel. If the fixed fixing device is rotated in the first direction of rotation D1, it is initially tensioned more strongly, until the fixing element reverts back again and bears against the main body, and the fixing device is thus released.

In accordance with a further embodiment, at least two resilient fixing elements arranged in a contact region each on the main body are provided, wherein the at least two fixing elements, in respect of their angle relative to the tangent on the main body running through the respective contact regions, are oriented in such a way that the fixing elements bear against the main body in the event of a rotation of the main body about the longitudinal axis of the main body in a first direction of rotation D1.

In accordance with a further embodiment, at least two resilient fixing elements arranged in a contact region each on the main body are provided, wherein the at least two fixing elements, in respect of their angle relative to the tangent on the main body running through the respective contact regions, are oriented in such a way that the fixing elements splay out from the main body in the event of a rotation of the main body about the longitudinal axis of the main body in a second direction of rotation D2.

In accordance with a further embodiment of the fixing device, the extent of the fixing element in a first direction of extent is greater than the extent of the fixing element in the second direction of extent. The length of the fixing elements in their second direction of extent, measured from their contact region to the edge opposite the contact region, can also be different for different fixing elements. In a development of the present invention, the thickness of the fixing elements may also vary. For example, the fixing elements on the side facing there contact region may have a greater thickness than on the edge of the fixing elements opposite the contact region.

In accordance with a further embodiment of the fixing device, the extent of the fixing element in the second direction of extent can reduce in the axial direction towards the end of the fixing element. As a result, the fixing element can better bear against the main body of the fixing device if the diameter of the blood vessel changes along the course of the blood vessel. This is advantageous when inserting the fixing device into a blood vessel or when removing the fixing device from a blood vessel. In particular, when removing the fixing device after a longer period of residence in the body, it is advantageous if the fixing device does not have any undercuts, so that the fixing element does not become hooked at incision points and the explantation can be performed without difficulty.

In accordance with a further embodiment of the fixing device, the extent of the fixing element in the second direction of extent can vary in the axial direction. Many vessels narrow or become wider along their longitudinal axis or have a specific course. By varying the length of the fixing element from its contact region to its edge opposite the contact region it is possible for the fixing device to be adapted to the geometry of the target vessel.

In accordance with a further embodiment of the fixing device, it is provided that the main body and the fixing element are made at least in part of liquid silicone rubber (LSR) and/or polyether-block-amide-block copolymer from the PEBAX® range by the company Arkema and/or of polyurethanes PU.

In accordance with a further embodiment of the fixing device, it is provided that the fixing element can comprise a stiffening element. The stiffening element of the fixing element can improve the bending moment of the fixing element. Particularly in the case of softer materials, such as silicone, it may be necessary—especially in the case of fixing devices for larger blood vessels—that the fixing element has to be stiffened so that it attains an appropriate tension for the fixing. Here, the stiffening element can comprise for example a mesh structure and/or a textile fabric and/or elements made of polyether ether ketone (PEEK) or polyurethanes (PU), such as ribs, bars, plates, strips, etc.

In accordance with a further embodiment of the fixing device, at least three fixing elements can be arranged symmetrically on the main body in such a way that the fixing elements are each arranged at equal distances from one another in the peripheral direction of the main body. As a result of this geometric, arrangement, the main body of the fixing device does not have any contact with the vessel wall, i.e. lies in the blood flow. For example, sensors can be anchored hereby in a blood vessel.

In accordance with a further embodiment of the fixing device, at least two fixing elements can be arranged on the main body asymmetrically in respect of the peripheral direction. Due to the asymmetric arrangement of the fixing elements, the main body of the fixing device, in addition to the fixing elements of the fixing device, is also connected to the wall of the blood vessel. As a result, for example, electrode contacts can be brought close to the wall of a blood vessel, for example, or—in the case of a particularly close arrangement of a fixing device to an electrode contact or in the case of an arrangement of a fixing device on both sides before and after an electrode contact, such as a ring electrode—can be pressed against the vessel wall. In this regard, reference is also made to parietal electrodes. The stimulation of the tissue or the sensing of bodily signals is hereby improved. For example, the parietal position of the electrodes allows a reduction of the pulse height of the stimulation pulses and therefore a more economical operation of the pulse generator, whereby the service life of the battery of the pulse generator can be extended.

In accordance with a further embodiment of the fixing device, the fixing element can have a structured surface on the side facing the main body. A structured surface can have a dimpled structure with dots, squares, diamonds, rectangles, etc., a surface comparable to the tread of a tire, a roughened surface, and a corrugated or ribbed structure. In the case of a structured surface that has a corrugated or ribbed structure, the corrugation peaks and valleys or ribs run along the second direction of extent of the fixing element. After the anchoring, the edge opposite the contact region bears against the blood vessel by means of the side of the fixing element originally facing the main body of the fixing device. The effect of the fixing is intensified by a structured surface. The orientation of the ribs/grooves, corrugation peaks/corrugation valleys transverse to the longitudinal axis of the fixing device increases the effect of the fixing along the longitudinal axis of the fixing device.

In accordance with a further embodiment of the fixing device, the fixing element can have a predetermined breaking point along the contact region between the fixing element and the main body. In the event of explantation of the fixing element, the fixing device can detach from the main body by the predetermined breaking point. The blood vessel is hereby prevented from being damaged by tensile forces that are exerted onto fixing elements that have become ingrown at incision points.

Furthermore, an implantable electrode lead having a lead body, at least one electrode for contacting bodily tissue, and a fixing device as described above is provided. In this case, the electrode lead may be an electrode lead for CRT therapy. The electrode lead can be configured here as a unipolar electrode lead, as a bipolar electrode lead, or as a multipolar electrode lead (3 or more electrodes). The fixing device can be formed here as part of the lead body. Alternatively, the fixing device can be configured as a tube which is slid over an electrode lead and for example is fastened to the electrode lead by means of a surgical thread. An electrode lead may contain one or more fixing devices as described above. Furthermore, an electrode lead can contain fixing devices as described above, wherein the multiple fixing devices can have different numbers of fixing elements.

In accordance with an embodiment of the implantable electrode lead, the fixing device is arranged on the electrode lead between two ring electrodes. Furthermore, the fixing device can be arranged directly proximally and/or distally of an electrode.

Additional features, aspects, objects, advantages, and possible applications of the present invention will become apparent from a study of the exemplary embodiments and examples described below, in combination with the Figures, and the appended claims.

### DESCRIPTION OF THE DRAWINGS

Further features, advantages and embodiments of the present invention will be described hereinafter with reference to the Figures, in which:

FIG. 1 shows an embodiment of a fixing device according to the present invention;

FIG. 2 shows a sectional illustration of a fixing device;

FIG. 3 shows a further sectional illustration of a fixing device;

FIG. 4 shows a fixing device inserted into a blood vessel;

FIG. 5A shows a sectional illustration of a fixing device, inserted into a catheter, with flattened fixing elements;

FIG. 5B shows a sectional illustration of a fixing device inserted into a blood vessel;

FIG. 5C shows a sectional illustration of a fixing device, inserted into a blood vessel, with tensioned fixing elements;

FIG. 6A shows a fixing device with a fixing element with stiffening element;

FIG. 6B shows a fixing device with a fixing elements which has a structured surface;

FIG. 6C shows a fixing device inch the fixing element has a predetermined breaking point;

FIG. 7 shows a distal region of an electrode lead on which there are arranged fixing devices;

FIG. 8 shows a fixing device which can be slid onto an electrode lead; and

FIG. 9 shows a fixing device with 4 fixing elements.

### DETAILED DESCRIPTION

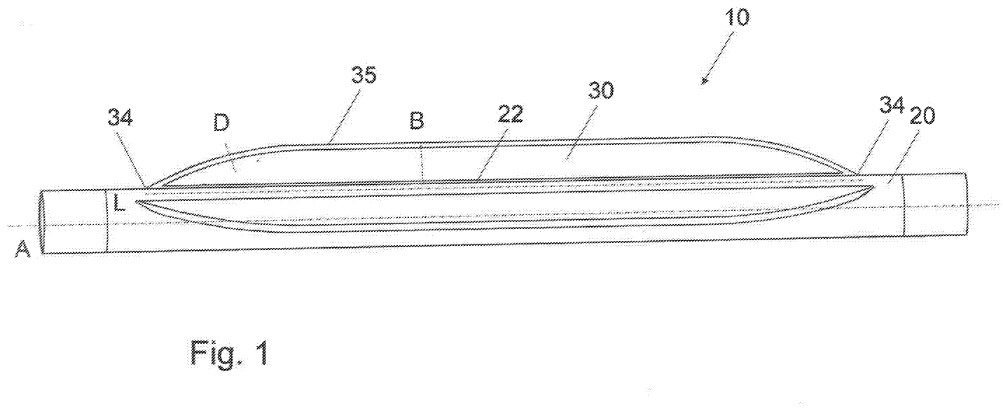

FIG. 1 shows a side view of a fixing device 10 according to the present invention with a cylindrical main body 20. Two fixing elements 30 are arranged on the main body 20 of the fixing device 10 by means of the two contact regions 22. In the case of the shown fixing device 10, the fixing elements 30 starting at the contact regions 22 run tangentially to the main body 20. The fixing elements 30 run in the axial direction A in a first direction of extent L. Furthermore, in a second direction of extent B the fixing elements 30 rim from the contact region 22 to the edge 35 of the fixing element 30 opposite the contact region 22. The length of the fixing element 30 in its second direction of extent B can vary along the longitudinal axis A of the fixing device 10. For example, the edge 35 of the fixing element 30 opposite the contact region 22 can approach the ends 34 of the fixing element 30 on the main body. Furthermore, the fixing element 30 can have a semi-circular or curved form.

The fixing elements 30 are made of a flexible material, such that the fixing elements 30 are flexible at least along their second direction of extent B. The thickness D of the fixing elements can vary both along their first direction of extent L and along the second direction of extent B. For example, the thickness D of the fixing element 30 can decrease, starting from the contact region 22 to the edge 35 of the fixing element 30 opposite the contact region 22. Likewise, the thickness D of the fixing element 30 can vary along the first direction of extent L. For example, it is conceivable that the thickness D at the ends 34 of the fixing element 30 is smaller than in the middle region of the fixing element 30. Likewise, the fixing element 30 can have a thickened framing along its outer edge, whereas a smaller thickness D is provided for the fixing element 30 in its inner region.

A cylindrical main body 20 is provided in the fixing device 10 according to FIG. 1. The main body 20, however, can also be configured in the form of a cone or in the form of a square, if this design of the main body 20 performs the same purpose as a cylinder or a cone in respect of the arrangement of the fixing element 30 on the fixing device 10. Furthermore, the main body 20 of the fixing device 10 can have one or more lumen(s) along its longitudinal axis A. The connection lines between the plug (not shown) of the electrode lead 100 and the electrodes 120 can run through the lumen or the lumens. Furthermore, the lumen or one of the lumens can receive a mandrel or guide wire introduced into the electrode lead 100.

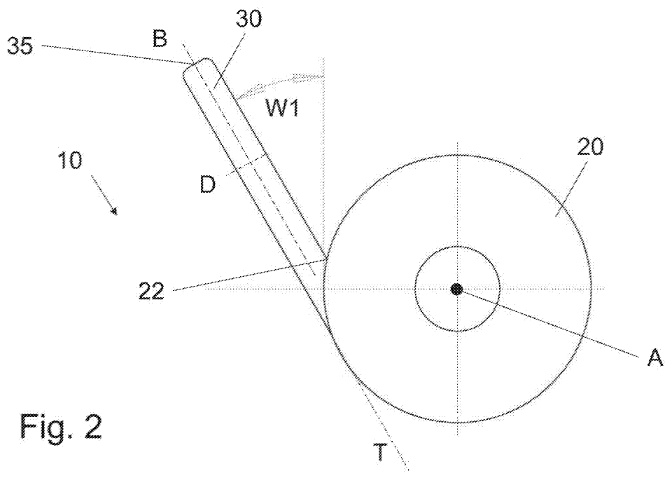

FIG. 2 shows a sectional illustration of a fixing device 10 with a fixing element 30. In this illustration, the second direction of extent B of the fixing element 30 lies in the drawing plane. In the sectional illustration, the fixing element 30 extends from the contact region 22, which connects the fixing element to the main body 20, to the edge 35 of the fixing element 30 opposite the contact region 22. In order to better explain the arrangement of the fixing element 30 on the main body 20, FIG. 2 additionally contains the tangent T on the circular main body 20, wherein the tangent T contacts the main body 20 at the contact region 22. The fixing element 30 is arranged on the main body 20 of the fixing device 10 such that it forms an angle W1 with the tangent T. Even if the angle W1 shown in FIG. 2 is greater than 0-degrees, the fixing element 30 can also form an angle of 0-degrees with the tangent T, that is to say the fixing element 30 can also be arranged parallel to the tangent T.

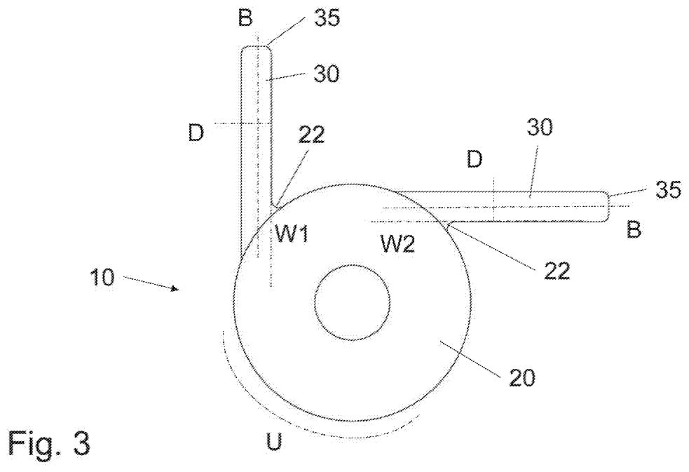

FIG. 3 likewise shows a sectional illustration of a fixing device 10, however, in this case two fixing elements 30 are provided on the main body 20 along the peripheral direction U. The two fixing elements 30 enclose the angle W1 or W2 by means of their respective tangents T. In principle, it is not necessary for both angles W1 and W2 to be identical. As already mentioned in the explanatory text for FIG. 2, one of the angles W1 or W2 also both angles W1 and W2 can be 0-degrees. One of the fixing elements 30 or also both fixing elements 30 can thus run parallel to the corresponding tangent T. Furthermore, the length of the fixing elements 30 along their respective second directions of extent B can be different.

The fixing devices 10 shown in FIGS. 1 to 3 with one or two fixing elements 30 represent exemplary fixing devices 10 which are suitable for bringing the main body 20 of the fixing device 10, in the state implanted in a blood vessel 50, into a position resting against the wall. The different lengths, provided optionally, of the fixing elements 30 in their respective second directions of extent B can be advantageous if a parietal position of the main body 20 in a blood vessel 50 of larger cross-section is sought.

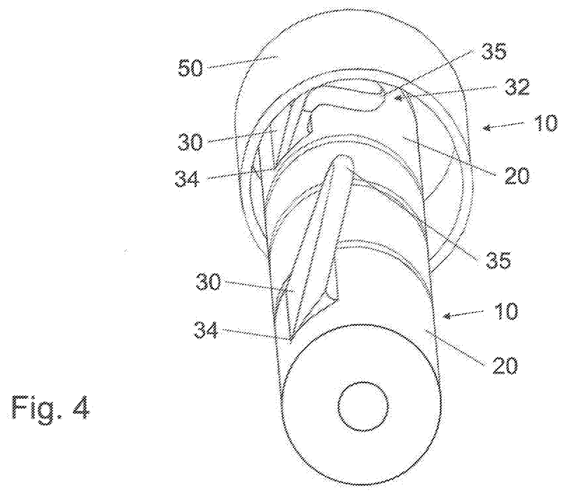

FIG. 4 is a schematic illustration of a blood vessel 50 with a fixing device 10 inserted partially into the blood vessel 50. The cross-section of the blood vessel 50 is smaller here than the circumference around the fixing device 10 which at the same time contacts the edges 35 of the relaxed fixing elements 30 in the main body 20 opposite the contact regions 20. The fixing elements 30 are pressed in the direction of the main body 20 of the fixing device 10 by the inner wall of the blood vessel 50. The fixing elements 30 and the main body 20 of the fixing device 10 therefore bear against the inner wall of the blood vessel 50 in the case of the shown fixing device 10. Depending on the ratio between the above-mentioned circumference around the fixing device 10 and the cross-section of the blood vessel 50, only the edges 35 of the fixing elements 30 contact the inner wall of the blood vessel 50, for example with diameters of the blood vessel 50 of comparative size, or, in the case of narrower vessels, larger regions of the fixing elements 30 bear against the inner wall of the blood vessel 50. The fixing elements 30 are hereby curved along their second direction of extent B.

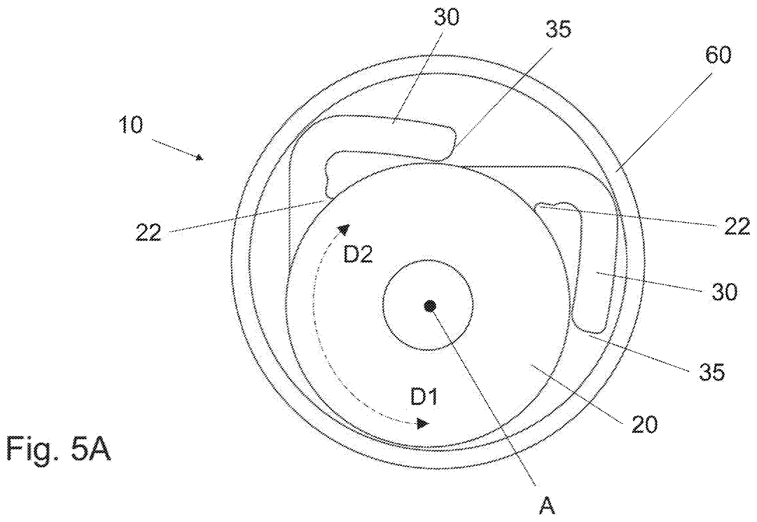

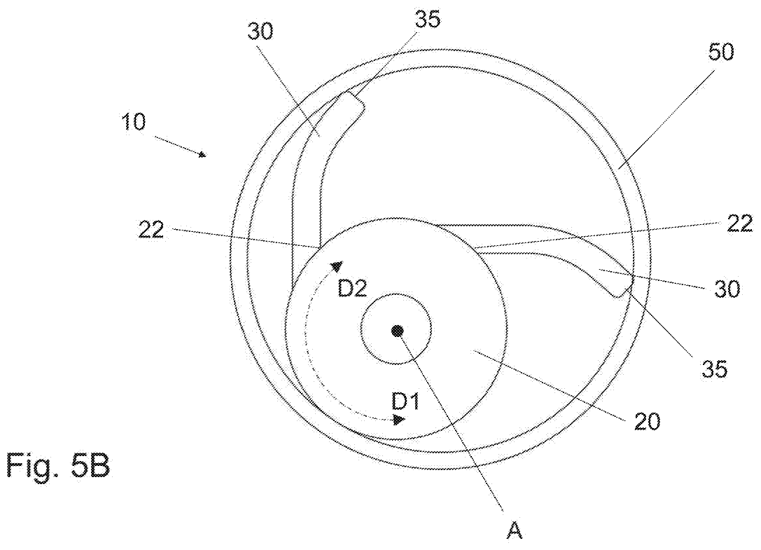

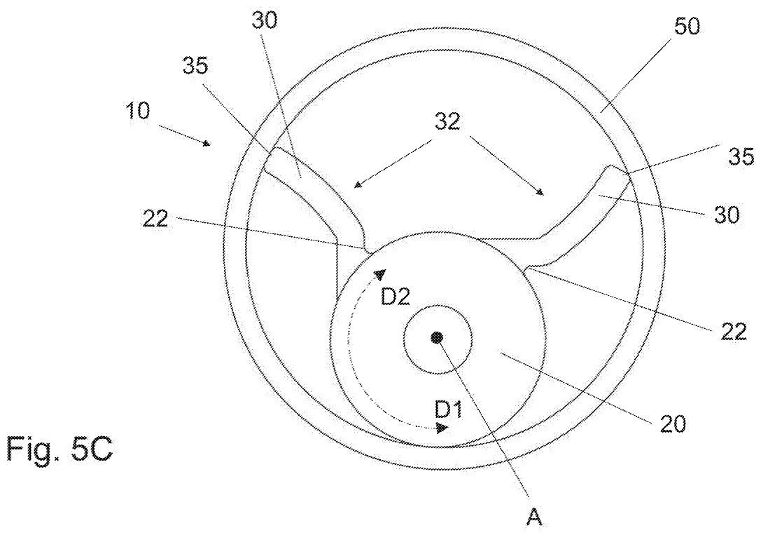

If, as in sectional illustration shown in FIG. 5A, a catheter 60 is used to insert the fixing device 10, the fixing elements 30 are pressed by the inner wall of the catheter 60 in the direction of the main body 20 of the fixing device 10 to such an extent that they bear against the main body 20. So that the fixing device 10 with fixing elements 30 flattened against the main body 20 has a round cross-section, recesses can be provided on the main body 20, which recesses can receive the fixing elements 30 flattened against the main body 20. As a result, with flattened fixing elements 30, the fixing elements 30 do not protrude beyond the main body 20. This is advantageous if the fixing device 10 is part of an electrode lead 100, since the diameter of the electrode lead is not hereby increased by the fixing device 10.

If the fixing device 10 is to be inserted into a blood vessel 50 or into a catheter 60 or is to be advanced from a blood vessel of larger cross-section into a vessel of smaller cross-section, it is thus important that the fixing element 30, on account of its elasticity, bends through the constriction in the direction of the main body 20. If the fixing element 30, at its ends 34, has flanks extending in a rectangular manner with respect to the main body, the fixing element 30, with a reduction of the inner diameter of a blood vessel 50 or a catheter 60, can thus only be guided with difficulty towards the smaller cross-section during the insertion process. It is therefore advantageous if, in the case of the fixing element 30, the extent in the second direction of extent B reduces in the axial direction A towards the end 34 of the fixing element 30. The fixing element 30 can hereby lie better against the main body 20 of the fixing device 10 if the diameter of the blood vessel 50 changes along its course. This is advantageous when inserting the fixing device 10 into a blood vessel 50 or when removing the fixing device 10 from a blood vessel 50. A possibility of improving the course of the edge 35 of the fixing element 30 lies in forming the flanks at the ends of the fixing element 30 in an acute angle relative to the main body 20 of the fixing device 10. Furthermore, the edge 35 of the fixing element 30 can be rounded at the ends 34 of the fixing element in the axial direction, as is indicated by way of example in FIG. 1.

FIG. 5B shows, in a sectional illustration, a fixing device 10 inserted into a blood vessel 50, the edges 35 of the fixing elements 30 of said fixing device, and the main body 20 of said fixing device bearing against the inner wall of the blood vessel 50. If the fixing device 10 is rotated about its longitudinal axis A in the first direction of rotation D1, a first force thus acts on the fixing element 30 due to the contact of the edges 35 and the main body 20 with the vessel wall and presses the fixing element 30 in the direction of the main body 20 of the fixing device 30. The fixing element 30 therefore bears against the main body 20 in the event of a rotation about the longitudinal axis A in the first direction of rotation D1.

FIG. 5C shows, in a sectional illustration, how the fixing device 10 is tensioned in the blood vessel 50. To this end, the fixing device 10 is rotated about its longitudinal axis A in the second direction of rotation D2. A second force, which deploys the fixing element 30, thus acts on the fixing element 30 on account of the contact of the edges 35 and of the main body 20 with the wall of the blood vessel 50. Due to the second force, the fixing element 30 sticks against the inner wall of the blood vessel 50 by means of its edge 35 opposite the contact region 22, and the fixing element 30 is hereby buckled and bent along the second direction of extent B, such that the fixing element 30 has a convex side and a concave side. Here, the convex side of the fixing element 30 points in the second direction of rotation D2, whereas the concave side of the fixing element 30 points in the first direction of rotation D1. A balance is established depending on the diameter of the blood vessel 50 and the bending moment of the fixing element 30 along the second direction of extent B. In this state the fixing device 10 is tensioned and the fixing element 30 exerts a force onto the wall of the blood vessel 50, whereby the fixing device 10 remains fixed in the blood vessel 50. If the fixing device 10 is rotated out of its fixed state in the first direction of rotation D1, it is initially tensioned more strongly, until the fixing element 30 reverts back again and bears against the main body 20, and the fixing device 10 is thus released.

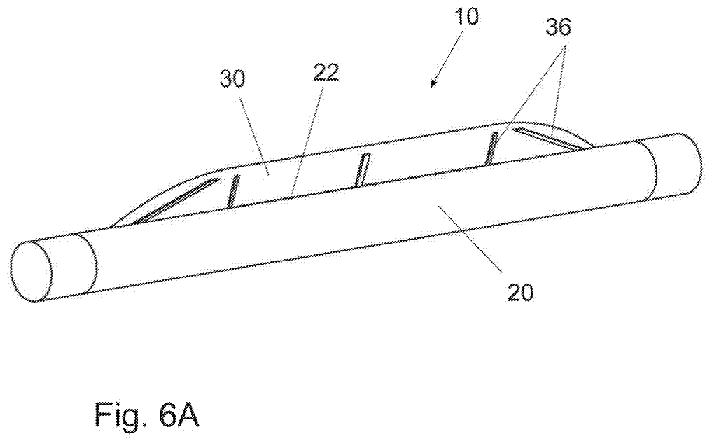

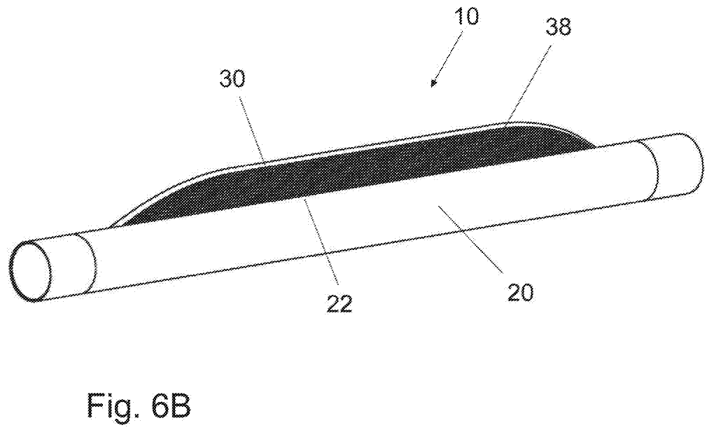

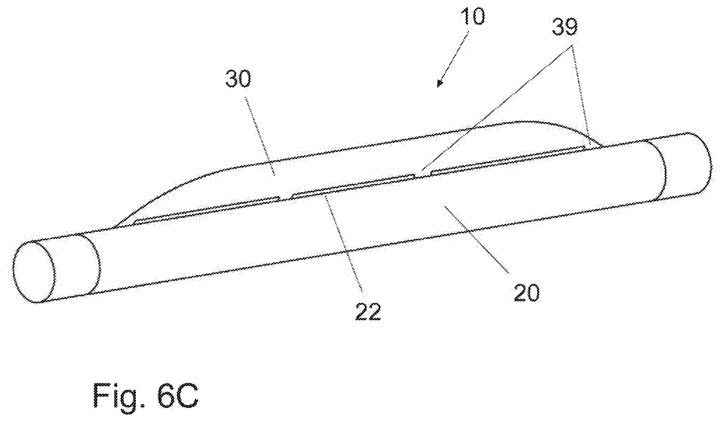

FIG. 6A shows a fixing device 10 with a fixing element 30, wherein the fixing element 30 has a stiffening element 36. The bending moment of the fixing element 30 can be improved by the stiffening element 36 of the fixing element 30. Particularly in the case of softer materials, such as silicone, it may be necessary—especially in the case of fixing devices for larger blood vessels—that the fixing element has to be stiffened so that it attains an appropriate tension for the fixing. Here, the stiffening element can comprise for example a mesh structure and/or a textile fabric and/or elements made of polyether ether ketone (PEEK) or polyurethanes (PU), such as ribs, bars, plates, strips, etc.

In FIG. 6B, it is shown that the fixing element 30 can have a structured surface 38 on the side facing the main body 20 of the fixing device 10. One possibility for providing a structured surface 38 lies in the forming of a corrugated or ribbed structure, wherein the corrugation peaks and valleys or ribs run along the second direction of extent B of the fixing element 30. After the anchoring of the fixing device 10, the edge 35 of the fixing element 30 opposite the contact region 22 bears against the blood vessel 50 by means of the side of the fixing element 30 facing the main body 20 of the fixing device 10. By means of a structured surface 38, the effect of the fixing in the axial direction A is intensified. The orientation of the ribs/grooves transverse to the longitudinal axis A of the fixing device 10 increases the fixing effect in the longitudinal direction A. Other structured surfaces 38, such as a dimpled structure or “tire profile” structure, are also conceivable.

In FIG. 6C, a fixing device 10 is shown, in which the fixing element 30 has a predetermined breaking point 39 along the contact region 22 between fixing element 30 and main body 20. As a result of this predetermined breaking point 39, the fixing element 30 can detach from the main body 20 in the event of an explantation of the fixing device 10. The blood vessel is hereby prevented from being damaged by tensile forces that are exerted onto fixing elements 30 that have become ingrown at incision points.

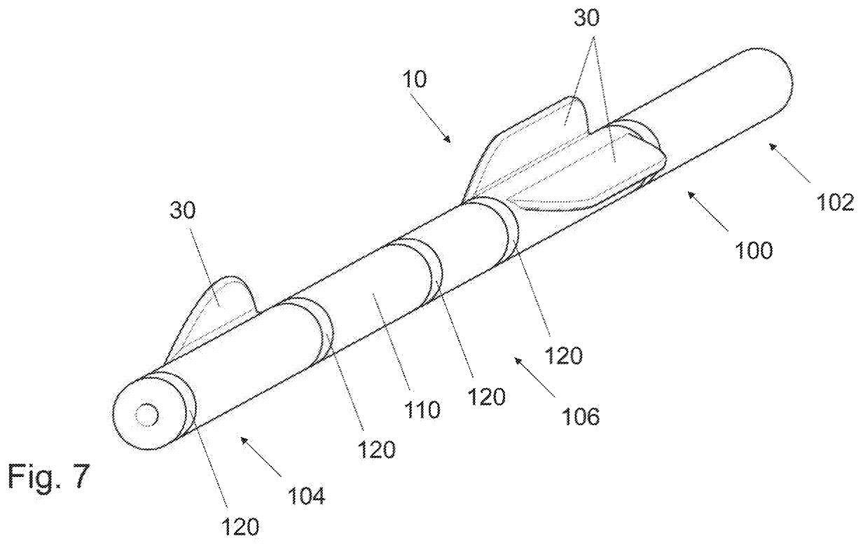

FIG. 7 shows the distal region 106 of a quadrupolar implantable electrode lead 100 having a lead body 110, four electrodes 120 formed as ring electrodes contacting bodily tissue, and a fixing device 10. The electrode lead 100 can be designed alternatively also as a unipolar electrode lead, as a bipolar electrode lead, or as a multipolar electrode lead (3 or more electrodes). The electrode lead 100 according to FIG. 7 can be, for example, an electrode lead for CRT therapy. The electrode lead 100 in this case has two fixing devices 10, wherein one of the two fixing devices 10 is arranged proximally of the electrode 120 which is provided closest to the proximal end 102 of the electrode lead. The second fixing device 10 is arranged between the first and the second ring electrode 120 at the distal end 104 of the electrode lead 100. Both fixing devices 10 are formed as part of the lead body 110 of the electrode lead 100. Both fixing devices 10, amongst other things, satisfy the purpose of bringing the electrode lead 100 within a blood vessel 50 into a parietal position and fixing it there.

Typical CRT electrode leads 100 have a diameter of approximately 1.6 mm. They must be inserted into blood vessels 50 having diameters of from approximately 1.8 mm to approximately 5 mm. For electrode leads 100 of this kind, fixing devices 10 with fixing elements 30 that have a length along the second direction of extent B of from 1.0 mm to 4 mm are advantageous. The fixing elements 30 preferably have lengths along their first direction of extent of from 3 mm to 40 mm, and more preferably from 5 mm to 20 mm. The fixing elements 30 for CRT electrode leads 100 of this kind preferably have a thickness D of from 0.15 mm to 0.5 mm, more preferably a thickness D of from 0.2 mm to 0.4 mm, and more preferably a thickness D of from 0.2 mm to 0.3 mm. Furthermore, in the case of CRT electrodes of this kind, a fixing device 10 is provided in which the angle W1, W2 that is formed by the fixing element 30 and the associated tangent T is smaller than 45-degrees, preferably smaller than 30-degrees, and more preferably smaller than 20-degrees.

Implantable electrode leads 100 with fixing devices 10 can also be used in other areas. For example, electrode leads 100 of this kind, with fixing device 10, can be operated on a neuro stimulator. The diameter of the vessel in which the fixing device 10 can be inserted and anchored is also not limited to 5 mm. Rather, in the case of larger vessel diameters, the dimensions of the fixing elements 30 can be adapted accordingly. In addition, in blood vessels 50 of larger diameter, electrode leads 100 of larger diameter than described further above for electrode leads 100 for CRT therapy can be used. The main body 20 of the fixing device 10 connected to an electrode lead 100 can thus also have a larger diameter D.

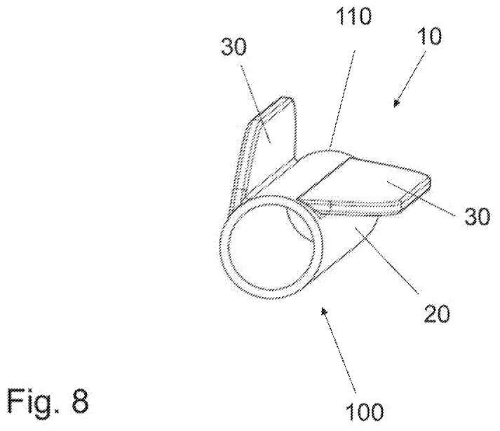

In FIG. 8 it is shown that the fixing device 10 can be configured alternatively as a tube which is slid over an electrode lead 100 and, for example, is fastened to the electrode lead by means of a surgical thread or a detent connection. In order to receive the surgical thread, the fixing device 10 can have one or more peripheral grooves. Other possibilities are also conceivable for the fixing of the fixing device 10 to the electrode lead 100. For example, a detent connection can be provided between the electrode lead 100 and fixing device 10. For example, a narrowed region or a constriction is provided on the lead body 110 of the electrode lead 100 for a detent connection of this kind. As counter piece to the narrowed portion on the lead body 110, a narrowed zone can be provided likewise in the lumen of the tubular fixing device 10, by means of which narrowed zone the fixing device 10 latches on the lead body 110. One or more fixing devices 10 can be fastened to an electrode lead 100 by means of one of the described mechanisms (e.g., surgical thread or detent connection).

Also conceivable are electrode leads 100 that have both variants of the fixing device 10, namely, fixing devices 10 that are part of the lead body 110 of the electrode lead 100 (i.e. were attached during the manufacture of the electrode lead) and fixing devices 10 that can be fastened to the lead body 110 of the electrode lead 100 subsequently. Furthermore, an electrode lead 100 can contain fixing devices 10, wherein the plurality of fixing devices 10 can have different numbers of fixing elements 30.

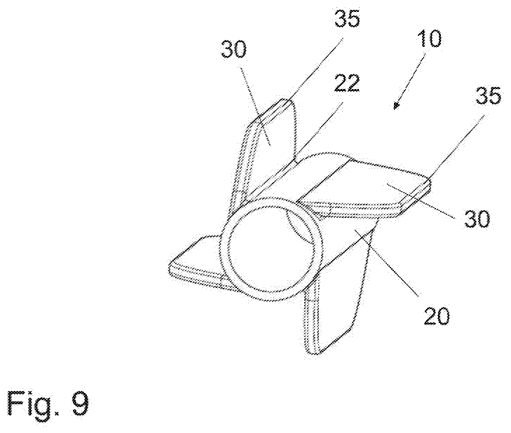

FIG. 9 shows a fixing device 10 with 4 fixing elements 30 that are mounted at equal distances from one another along the circumferential direction U of the main body 20. With fixing devices 10 of this kind an electrode lead 100 can be fixed in a blood vessel 50 without the electrode lead coming into contact with the inner wall of the vessel. A symmetrical arrangement of this kind of the fixing elements 30 on the main body 20 is achievable already with 3 fixing elements. Fixing devices 10 having 5, 6, 7, 8, 9, etc. fixing elements 30 are also conceivable. The maximum number of fixing elements 30 is dependent on the diameter of the fixing device 10 and the selected length along the second direction of extent B of the fixing elements 30.

It will be apparent to those skilled in the art that numerous modifications and variations of the described examples and embodiments are possible in light of the above teachings of the disclosure. The disclosed examples and embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A fixing device for fixing an implantable electrode lead in a blood vessel, comprising:
a main body which has a circular cross-section; and
at least one resilient fixing element arranged on the main body in a contact region;
wherein the fixing element extends in a first direction of extent in an axial direction in respect of the main body,
wherein the fixing element in a second direction of extent extends away from the main body starting from the contact region, and
wherein the fixing element encloses an angle of <45-degrees with a tangent on the main body running through the contact region.

2. The fixing device according to claim 1, wherein at least two resilient fixing elements are arranged in a contact region each on the main body, wherein the at least two fixing elements, in respect of their angle relative to the tangents on the main body running through the respective contact regions, are oriented in such a way that the fixing elements bear against the main body in the event of a rotation of the main body about the longitudinal axis of the main body in a first direction of rotation.

3. The fixing device according to claim 1, wherein at least two resilient fixing elements are arranged in a contact region each on the main body, wherein the at least two fixing elements, in respect of their angle relative to the tangents on the main body running through the respective contact regions, are oriented in such a way that the fixing elements splay out from the main body in the event of a rotation of the main body about the longitudinal axis of the main body in a second direction of rotation.

4. The fixing device according to claim 1, wherein the extent of the fixing element in the first direction of extent is greater than the extent of the fixing element in the second direction of extent.

5. The fixing device according to claim 1, wherein the extent of the fixing element in the second direction of extent reduces in the axial direction towards the end of the fixing element.

6. The fixing device according to claim 1, wherein the main body and the fixing element are made at least in part of liquid silicone rubber and/or of PEBAX and/or of polyurethanes.

7. The fixing device according to claim 1, wherein the fixing element comprises a stiffening element,

8. The fixing device according to claim 7, wherein the stiffening element comprises a mesh structure and/or a textile fabric and/or elements made of PEEK or polyurethanes.

9. The fixing device according to claim 1, wherein at least three fixing elements are arranged symmetrically on the main body in such a way that the fixing elements each have equal distances from one another in the circumferential direction of the main body.

10. The fixing device according to claim 1, wherein at least two fixing elements are arranged on the main body asymetrically in respect of the circumferential direction.

11. The fixing device according to claim 1, wherein the fixing element has a structured surface on the side facing the main body.

12. The fixing device according to claim 11, wherein the structured surface of the fixing element can be configured in the form of a dimpled structure consisting of dots and/or squares and/or diamonds and/or rectangles and/or a surface comparable to the tread of a tire and/or a roughened surface and/or a corrugated or ribbed surface.

13. The fixing device according to claim 1, wherein the fixing element has a predetermined breaking point along the contact region between the fixing element and the main body.

14. An implantable electrode lead having a lead body, at least one electrode for contacting bodily tissue, and a fixing device according to claim 1.

15. The implantable electrode lead according to claim 14, wherein the fixing device is arranged between two ring electrodes.
